(19) United States
(12) Patent Application Publication
(10) Pub. No.: US 2020/0232969 A1
(43) Pub. Date: Jul. 23, 2020

(54) DETECTION DEVICE BASED ON SHADOW IMAGING OF PLATELETS, AND METHOD THEREOF

(71) Applicant: NANJING UNIVERSITY, Nanjing, Jiangsu (CN)

(72) Inventors: Feng YAN, Nanjing, Jiangsu (CN); Cheng YANG, Nanjing, Jiangsu (CN); Lian WANG, Nanjing, Jiangsu (CN); Limin ZHANG, Nanjing, Jiangsu (CN); Xia HUA, Nanjing, Jiangsu (CN); Haowen MA, Nanjing, Jiangsu (CN); Xiaofeng BU, Nanjing, Jiangsu (CN); Xu CAO, Nanjing, Jiangsu (CN)

(21) Appl. No.: 16/652,605

(22) PCT Filed: Mar. 23, 2018
(86) PCT No.: PCT/CN2018/080171

§ 371 Date: Mar. 31, 2020

(30) Foreign Application Priority Data
Oct. 11, 2017 (CN) 201710941056.0

(51) Int. Cl. G01N 33/49; G02B 21/00; B01L 3/00

(52) U.S. Cl. CPC G01N 33/4905; B01L 3/50273; G02B 21/008; G02B 21/0008

(57) ABSTRACT

The present invention provides a detection device based on the shadow imaging of platelets and a method thereof. The method comprises following steps: optically projecting and/or photographing a blood sample to be detected directly by an image sensor chip having a submicron pixel size and a megapixel scale, the blood sample being injected into a sample chamber of a microfluidic chip fixed to the surface of the image sensor chip; and then, identifying and counting imaging results by an image processing algorithm when the physical pixel size, in the imaging results, occupied by abnormally-sized platelets in the blood sample is significantly greater than the physical pixel size occupied by normally-sized platelets, so as to obtain the number and proportion of the abnormally-sized platelets. The present invention compensates for the defects of existing optical lens-based microscopic detection, provides a large field of view while satisfying the needs for resolution, greatly improves the detection efficiency, can realize the statistically significant microscopic observation, and provides early warning and diagnostic reference for the occurrence of clinical diseases such as stroke.

### TECHNICAL FIELD OF THE INVENTION

The present invention relates to a detection device based on the shadow imaging of platelets and a method thereof, in particular to a microscopic shadow imaging device based on an image sensor chip with a super-small pixel size and a super-large pixel scale, which is used with a microfluidic chip to detect the number and proportion of abnormally-sized platelets in a blood sample and provides early warning and diagnostic reference for the occurrence of related clinical diseases such as stroke.

### BACKGROUND OF THE INVENTION

As people's living habits and dietary habits change, stroke has become the “leading cause” of the death among residents in China. Stroke, commonly known as “cerebral apoplexy”, has the characteristics of high incidence rate, high death rate and high disability rate. Stroke is classified into ischemic stroke and hemorrhagic stroke. The ischemic stroke has a higher incidence rate than the hemorrhagic stroke, accounting for 60% to 70%. Occlusion and stenosis of the internal carotid artery and vertebral artery may lead to ischemic stroke. Some medical studies have shown that the ischemic stroke is resulted from the blockage of blood vessels due to the formation of blood clots in blood vessels. During the formation of blood clots, platelets aggregate in human blood when being activated. Therefore, statistical detection of the number and proportion of activated platelets in human blood is of great reference significance for early warning and diagnosis of ischemic stroke.

Microscopic objects above micron scale, such as platelets in human blood, are usually observed by a conventional optical microscope. Platelets in human blood are about 2 μm to 4 μm in diameter in normal circumstances. They may be activated to grow filamentous pseudopods when some pathological changes occur in the human body. In this case, the platelets are 8 μm to 25 μm in diameter. Conventionally, platelets in a blood sample are amplified and imaged by an optical microscope using an optical lens. Due to the use of the lens, the detection device using this detection method is relatively large in size. In addition, due to the restrictions to its operating mechanism, the conventional optical lens microscopy is unable to provide a large field of view while providing a high resolution, leading to long statistical detection time and high cost, and is thus inapplicable to such statistically significant observation.

Accordingly, the statistical detection of abnormally-sized platelets in human blood requires a detection method and a corresponding optical microscopic imaging device, which realize simple structure, convenient operation, and large field of view while providing certain resolution.

### SUMMARY OF THE INVENTION

In view of the deficiencies in the prior art, an objective of the present invention is to provide a detection device based on the shadow imaging of platelets and a method thereof, which realize the shadow imaging of platelets in a blood sample directly by an image sensor chip having a submicron pixel size and a megapixel scale, provide a large field of view while satisfying the needs for resolution, greatly improve the detection efficiency and can realize the statistically significant microscopic observation.

The device provided by the present invention employs the following technical solutions.

A detection device based on the shadow imaging of platelets is provided, including a shadow imaging device, an LED light source, a chip control system, a data storage and processing system and a data display system, wherein the shadow imaging device includes an image sensor chip and a microfluidic chip; the image sensor chip has a megapixel scale and a submicron pixel size; the microfluidic chip is fixed to the surface of the image sensor chip; the surface of the image sensor chip serves as a substrate of the microfluidic chip; a cavity on the microfluidic chip and the surface of the image sensor chip form a sample chamber; the LED light source is arranged directly above the shadow imaging device, and a light emitting surface of the LED light source is located on an optical axis of the shadow imaging device and covers the surface of the whole image sensor chip; the chip control system is connected to the image sensor chip to drive and control the operation and data readout of the image sensor chip; the data storage and processing system is connected to the image sensor chip to calculate and process data transmitted by the image sensor chip; and, the data display system is connected to the data storage and processing system to display the processed data result.

The method provided by the present invention employs the following technical solutions.

A detection method based on the shadow imaging of platelets is provided, including following detection steps:

step 1: fixing a microfluidic chip to the surface of an image sensor chip to form a sample chamber, and injecting a proper amount of human blood sample to be detected into the sample chamber;

step 2: illuminating the human blood sample placed in the sample chamber by using an LED light source as a lighting source of a shadow imaging device, and optically projecting and/or photographing the blood sample to be detected directly by the image sensor chip to obtain a projected image of the human blood sample, the physical pixel size occupied by a directly-projected image of abnormally-sized platelets on the image sensor chip being about 8 μm to 25 μm, and the physical pixel size occupied by a directly-projected image of normally-sized platelets being about 2 μm to 5 μm; and

step 3: statistically analyzing the shadow imaging results of the human blood sample in the step 2, and obtaining the number and proportion of the abnormally-sized platelets in the human blood sample in unit volume directly by an image processing algorithm according to the shadow imaging results since the physical pixel size occupied by the abnormally-sized platelets is significantly greater than the physical pixel size occupied by the normally-sized platelets.

The method and device provided by the present invention have the following beneficial effects.

(1) Any detection method using an optical lens system is not needed, so that the complexity of the system is reduced, the rapid and simple detection of abnormally-sized platelets in a blood sample is realized, and it is of great reference significance for early warning and diagnosis of clinical diseases such as stroke.

(2) With the solutions of the present invention, high resolution and large-field-of-view imaging are combined perfectly. Since the resolution in this detection method depends on the pixel size of the image sensor chip and the field of view depends on the pixel integration scale of the image sensor, a large field of view can be achieved while realizing a high resolution. Thus, the time for statistical detection is shortened, the cost is reduced and the statistically significant microscopic observation is realized.

### BRIEF DESCRIPTION OF THE DRAWINGS

FIG. 1 is a schematic view of an image sensor chip having a submicron pixel size and a megapixel scale according to an embodiment of the present invention;

FIG. 2 is a schematic structure diagram of a composite dielectric gate photosensitive detector according to an embodiment of the present invention;

FIG. 3 is a schematic structure diagram of a semi-floating gate transistor according to an embodiment of the present invention.

FIG. 4 is a schematic structure diagram of a microfluidic chip when viewed from the front side according to an embodiment of the present invention;

FIG. 5 is a schematic structure diagram of the microfluidic chip when viewed from the back side according to an embodiment of the present invention;

FIG. 6 is a schematic view of three sample chambers of different shapes on the microfluidic chip, in which (a) shows an elliptic sample chamber, (b) shows a circular sample chamber and (c) shows a fusiform sample chamber;

FIG. 7 is a schematic view of a device for early warning of stroke based on the shadow imaging of platelets; and

FIG. 8 is a diagram showing an example of a shadow imaging result of abnormally-sized platelets in a blood sample according to an embodiment of the present invention, where the right picture is an enlarged view of the box region in the left picture.

### DETAILED DESCRIPTION OF THE INVENTION

An embodiment of the present invention provides a detection device based on the shadow imaging of platelets, including: an image sensor chip 2 having a submicron pixel size and a megapixel scale and configured to record a two-dimensional shadow imaging result of a blood sample; a microfluidic chip 3 used as a space for accommodating a human blood sample and configured to accommodate a human blood sample to be detected and arrange the human blood sample to be detected in a single layer, the microfluidic chip 3 being directly attached to the surface of the image sensor chip 2; an LED light source 7 used as a lighting source of the whole imaging device, the LED light source 7 being arranged directly above the whole shadow imaging device, a light emitting surface of the LED light source 7 being located on an optical axis of the whole shadow imaging device and covering the surface of the whole image sensor chip 2; an image sensor chip control system 9 configured to drive and control the operation and data readout of the image sensor chip 2 having the submicron pixel size and the megapixel scale; a data storage and processing system 10 configured to calculate and process data transmitted by the image sensor chip 2 having the submicron pixel size and the megapixel scale; and, a data display system 11 configured to display the processed data result. The shadow imaging is defined relative to far-field optical imaging requiring an optical lens in a general sense. The conventional far-field optical imaging includes imaging using a microscope and various optical lenses. The shadow imaging belongs to the most basic lens-less imaging, i.e., imaging without using any optical lens.

The LED light source 7 is arranged directly above the whole shadow imaging device, and has a distance of 5 mm to 20 mm to the image sensor chip 2. The distance D (1 μm≤D≤500 μm) from the human blood sample to an actual light-sensing region of the image sensor chip 2 is in a submicron scale. Then, the image sensor chip 2 directly records the two-dimensional shadow of the human blood sample. In this way, the system becomes simple and convenient. Due to the quite short distance D (1 μm≤D≤500 μm) from the human blood sample to the actual light-sensing region of the image sensor chip, the field of view for shadow imaging is approximately equal to the size of the light-sensing region of the image sensor chip, and the amplification factor is slightly greater than 1. That is, the size of the shadow imaging result is slightly greater than the size of the actual sample. Thus, the size of the shadow imaging result can be regarded as the same as the size of the actual sample.

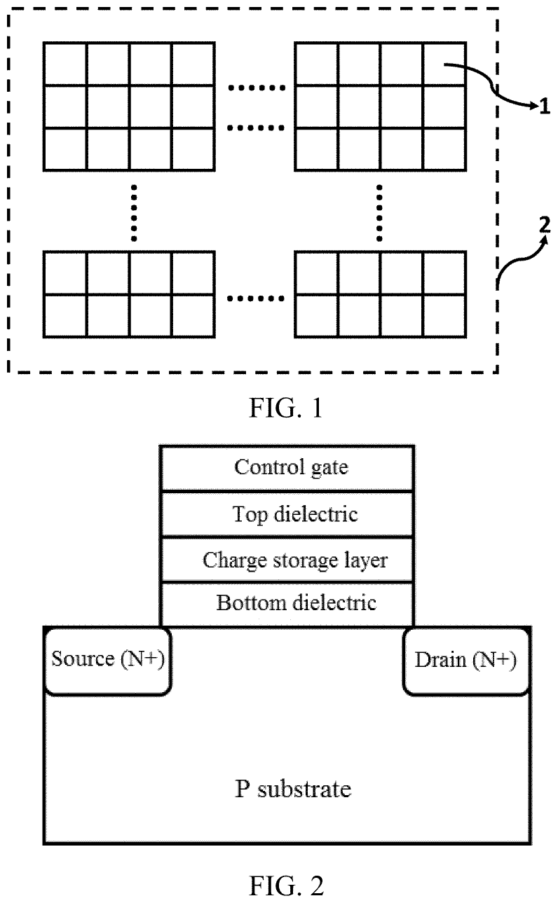

FIG. 1 is a schematic view of the image sensor chip 2 having a submicron pixel size and a megapixel scale according to this embodiment. The image sensor chip 2 includes multiple image sensors 1 in a submicron pixel size. The number of the image sensors 1 in the submicron pixel size is the pixel scale of the image sensor chip 2 having the submicron pixel size and the megapixel scale. The image sensor chip 2 may use semi-floating gate transistors or composite dielectric gate photosensitive detectors as pixel units. Since the platelets in the blood sample have a minimum diameter of 2 μm to 4 μm, the size of a single image sensor should be less than or equal to 1 μm×1 μm, and the whole image sensor chip should have 25 million or more pixels. Thus, when the pixel size is smaller, the resolution is higher, and finer sample details can be observed. Moreover, a super-large pixel scale ensures a large field of view while providing a high resolution. Therefore, statistically significant microscopic observation can be realized.

The composite dielectric gate photosensitive detector may be the composite dielectric gate photosensitive detector described in U.S. Pat. No. 8,604,409. As shown in FIG. 2, the photosensitive detector includes a semiconductor substrate (P-type). A bottom insulating dielectric layer, a photo-charge storage layer, a top insulating dielectric layer and a control gate are successively stacked above the semiconductor substrate. An N-type source and an N-type drain are formed on the semiconductor substrate (close to two sides of the stacked dielectric) by doping by ion implantation. Even according to the current technological level, it is very easy to manufacture such composite dielectric gate photosensitive detectors that are less than or equal to 1 μm in size. With the optimization of technological conditions, a single pixel size can be in 100 nanometer level, and the whole image sensor chip can easily have 100 million pixels.

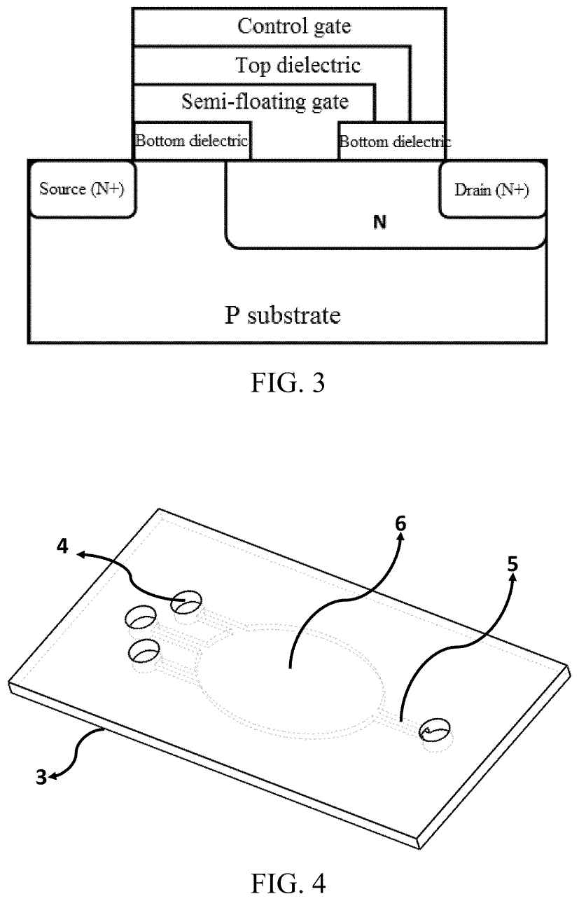

The semi-floating gate transistor may be, for example, the semi-floating gate transistor described in the literature (Wang P, Lin X, Liu L, et al. A semi-floating gate transistor for low-voltage ultrafast memory and sensing operation. [J]. Science (New York, N.Y.), 2013, 341(6146):640-643). As shown in FIG. 3, the photosensitive detector includes a semiconductor substrate (P-type). An N+ source is formed on the semiconductor substrate by ion implantation, and a large N-type drain is formed by two-step ion implantation. A bottom dielectric layer, a semi-floating gate, a top dielectric layer and a control gate are successively stacked above the semiconductor substrate. A trench is formed in the middle of the bottom dielectric layer by etching, so that the semi-floating gate comes into direct contact with the drain. Even according to the current technological level, it is very easy to manufacture such semi-floating gate transistors for photosensitive detection, which are less than or equal to 1 μm in size.

The microfluidic chip 3 may be made from glass or organic polymer. The organic polymer includes PDMS (polydimethylsiloxane), PMMA (polymethyl methacrylate), PC (polycarbonate) and hydrogel, epoxy resin or the like. The material for the whole microfluidic chip 3 should be quite high in light transmittance, should not affect the shadow imaging of the blood sample, and should be relatively low in hardness so that it is convenient to attach the microfluidic chip 3 to the image sensor chip 2 tightly to prevent the microfluidic channel 5 and the sample chamber 6 from liquid leakage.

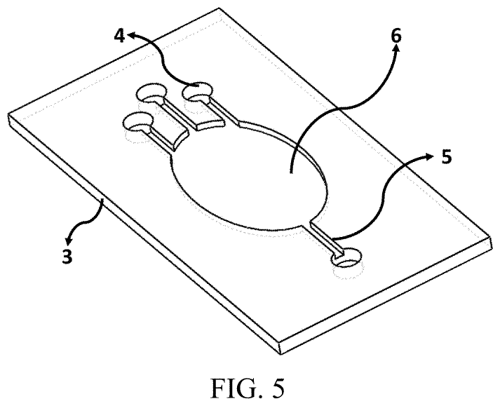

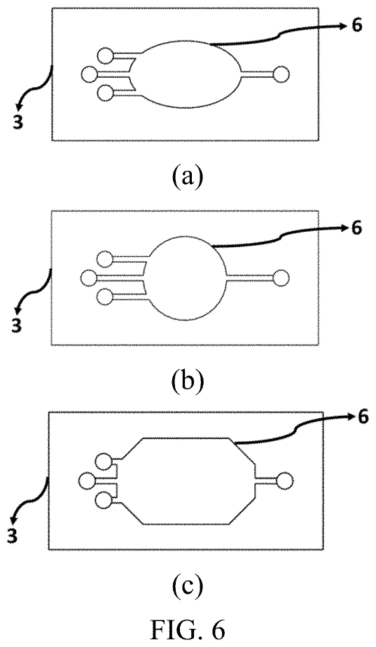

FIGS. 4 and 5 are schematic structure diagrams of the microfluidic chip 3 when viewed from the front side and the back side, respectively. The back side of the microfluidic chip 3 is directly attached to the surface of the image sensor chip 2. The microfluidic chip 3 includes one or more liquid inlets 4, one or more microfluidic channels 5 and one sample chamber 6. The height of the sample chamber 6 and the microfluidic channel 5 in a direction perpendicular to the surface of the image sensor chip 2 is Z (1 μm≤Z≤50 μm). The restriction to the height in this direction ensures that most platelets in the blood sample are arranged in a single layer, thereby preventing the arrangement of platelets in multiple layers. The arrangement of platelets in multiple layers is reflected as superposed shadows on the shadow imaging result, thereby affecting the accuracy of data processing. The sample chamber 6 may have different shapes. As shown in FIG. 6, the sample chamber 6 is elliptic, circular or fusiform. In such a structural design, the presence of air bubbles in the sample chamber can be avoided after the injection of the blood sample, thereby avoiding the influence on the subsequent shadow imaging.

There is no substrate in the microfluidic chip 3 in the present invention. Generally, the bottom of the microfluidic chip 3 will be sealed by using, for example, a glass sheet as the basal plate. Since the microfluidic chip 3 in this embodiment is relatively low in hardness, the microfluidic chip 3 can be directly attached to the surface of the image sensor 2 tightly when in use. The surface of the image sensor chip 2 serves as the substrate of the microfluidic chip 3, and the both are integrated to form the sample chamber 6. In this way, no liquid leakage will be caused. Moreover, the distance D (1 μm≤D≤500 μm) from the blood sample to the actual light-sensing region of the image sensor chip 2 becomes smaller. It is beneficial for the imaging resolution and signal-to-noise ratio of shadow imaging.

The LED light source 7 may be a narrowband LED light source having a central wavelength within a visible light region (400 nm to 700 nm) and a bandwidth of 5 nm to 10 nm. The LED light source 7 may also be formed by coupling a broadband LED light source with a single-mode optical fiber, the broadband LED light source having a central wavelength within the visible light region (400 nm to 700 nm) and a bandwidth of 10 nm to 35 nm, and the single-mode optical fiber having a diameter of 30 μm to 250 μm.

This embodiment provides a method using the detection device, including following specific steps.

Step 1: A proper amount (0.001 ml to 0.1 ml) of human blood sample 8 to be detected is injected into a sample chamber 6 of a microfluidic chip 3 attached to the surface of an image sensor chip 2.

The human blood sample 8, as a target to be detected by the shadow imaging device, may be platelet suspension and diluent thereof obtained by separation of human whole blood. For example, the human whole blood sample can be separated for a proper period of time (e.g., 5 min to 10 min) at a proper rotation speed (e.g., 1000 r/min) by a centrifugal machine to obtain platelet suspension with a very high purity and almost no other blood cells, and the platelet suspension can be subsequently diluted with a saline solution to obtain a platelet diluent with a desired concentration.

The injection of the human blood sample into the sample chamber 6 of the microfluidic chip 3 may be manual injection using a pipette, an injector or the like, or automatic injection using an injection pump. For example, it is possible to manually measure a proper amount of the blood sample 8 by a pipette or an injector and slowly inject the blood sample 8 by aligning the tip or needle to the liquid inlet of the microfluidic chip 3. It is also possible to measure a proper amount of the blood sample 8 by an injector and slowly inject the blood sample by an injection pump by connecting a pipe made from plastics, rubber or the like to the liquid inlet 4 of the microfluidic chip 3. In the process of injecting the blood sample 8 from the liquid inlet at one end into the microfluidic channel and finally into the sample chamber 6, air in the microfluidic chip 3 is slowly discharged from the liquid inlet at the other end, ensuring that the whole sample chamber is full of the blood sample without any air bubble.

Step 2: The human blood sample 8 placed in the microfluidic chip 3 is illuminated by using a narrowband LED light source 7 as a lighting source of a lens-less microscopic imaging system, and a projected image of the human blood sample 8 is obtained by the image sensor chip 2.

Step 3: The shadow imaging results of the human blood sample 8 obtained in the step 2 are statistically analyzed. The physical pixel size occupied by a directly-projected image of abnormally-size platelets 12 on the image sensor chip 2 is about 8 μm to 25 μm, and similarly, the physical pixel size occupied by a directly-projected image of normally-size platelets 13 is about 2 μm to 4 μm. Since the physical pixel size occupied by the abnormally-sized platelets 12 is significantly greater than the physical pixel size occupied by the normally-sized platelets 13, the number and proportion H of the abnormally-sized platelets in the human blood sample in unit volume can be obtained directly according to the shadow imaging results by an image processing algorithm (e.g., a basic edge detection algorithm). The result of data processing is transmitted to a data display system 11 (e.g., a liquid crystal display screen) to be displayed. Early warning and diagnostic reference are provided for the occurrence of clinical diseases such as stroke.

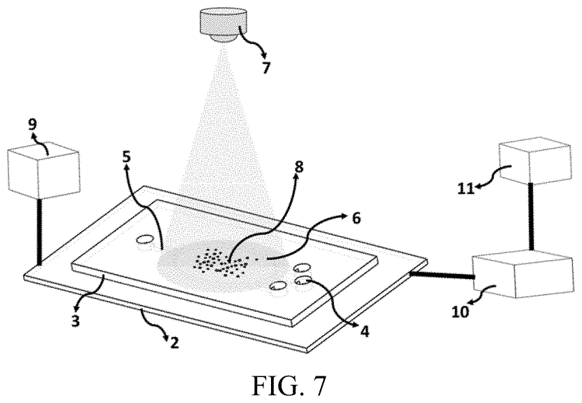

The implementation of the detection method provided in this embodiment will be described below with reference to FIG. 7.

(1) 1 ml of human whole blood is prepared and added with an anticoagulant and a diluent and then treated for 5 min at 1000 r/min by a centrifugal machine to obtain platelet suspension, and the platelet suspension is diluted to obtain a blood sample 8, i.e., platelet diluent.

(2) As shown in FIG. 7, 0.01 ml of the blood sample 8 is measured by a pipette and then manually injected, slowly, into the liquid inlet 4 of the microfluidic chip 3 and then into the sample chamber 6 through a microfluidic channel until the whole sample chamber is full of the blood sample 8. The blood sample 8 is arranged in a single layer.

(3) The narrowband LED light source 7 is turned on. The narrowband LED light source 7 is directly arranged above the whole shadow imaging device, and the light emitting surface of the narrowband LED light source 7 is located on the optical axis of the whole shadow imaging device. The narrowband LED light source 7 has a distance of 10 mm to the image sensor chip 2 having a super-small pixel size and a super-large pixel scale, so that the light emitting surface of the narrowband LED light source 7 covers the surface of the whole image sensor chip 2.

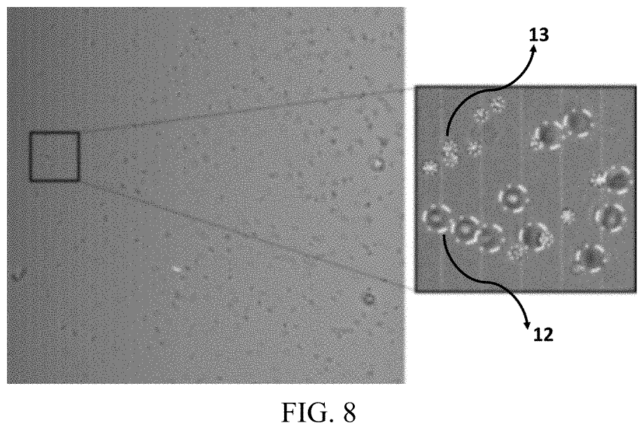

(4) The image sensor chip control system 9 is activated. Here, an FPGA control system is used to drive the image sensor chip 2 to acquire two-dimensional projected image data of the blood sample 8 and transmit the two-dimensional projected image data to the data storage and processing system 10 for statistical analysis. Here, host computer software on the computer side is used to obtain the number and proportion H of abnormally-sized platelets 12 in the blood sample 2 in unit volume, and the data is displayed on the data display system 11. Here, a liquid crystal screen is used. The schematic view of data result display is shown in FIG. 8. The physical pixel size occupied by the normally-sized platelets 13 is obviously different from the physical pixel size occupied by the abnormally-sized platelets 12.

It is to be noted that, the foregoing embodiments are not intended to limit the protection scope of the present invention, and any equivalent alternations or replacements made on the basis of the above technical solutions shall fall into the protection scope of the appended claims of the present invention.

1. A detection device based on the shadow imaging of platelets, characterized in that the device comprises a shadow imaging device, an LED light source, a chip control system, a data storage and processing system and a data display system; the shadow imaging device comprises an image sensor chip and a microfluidic chip; the image sensor chip has a megapixel scale and a submicron pixel size; the microfluidic chip is fixed to the surface of the image sensor chip; the surface of the image sensor chip serves as a substrate of the microfluidic chip; a cavity on the microfluidic chip and the surface of the image sensor chip form a sample chamber; the LED light source is arranged directly above the shadow imaging device, and a light emitting surface of the LED light source is located on an optical axis of the shadow imaging device and covers the surface of the whole image sensor chip; the chip control system is connected to the image sensor chip to drive and control the operation and data readout of the image sensor chip; the data storage and processing system is connected to the image sensor chip to calculate and process data transmitted by the image sensor chip; and, the data display system is connected to the data storage and processing system to display the processed data result.

2. The detection device based on the shadow imaging of platelets according to claim 1, characterized in that the image sensor chip uses semi-floating gate transistors or composite dielectric gate photosensitive detectors as pixel units, each of the pixel units has a size less than or equal to 1 μm×1 μm, and the whole image sensor chip has 25 million or more pixels.

3. The detection device based on the shadow imaging of platelets according to claim 1, characterized in that the microfluidic chip is directly attached to the surface of the image sensor chip, and the microfluidic chip is made from glass and organic polymer.

4. The detection device based on the shadow imaging of platelets according to claim 1, characterized in that a liquid inlet and a microfluidic channel are further formed on the microfluidic chip and the microfluidic channel is communicated with the cavity.

5. The detection device based on the shadow imaging of platelets according to claim 1, characterized in that the cavity on the microfluidic chip is elliptic, circular or fusiform.

6. The detection device based on the shadow imaging of platelets according to claim 1, characterized in that the sample to be detected in the sample chamber is arranged in a single layer; the distance D from the sample to be detected in the sample chamber to an actual light-sensing region of the image sensor chip is greater than or equal to 1 μm and less than or equal to 500 μm; and the height Z of the sample chamber in a direction perpendicular to the surface of the image sensor chip is greater than or equal to 1 μm and less than or equal to 50 μm.

7. The detection device based on the shadow imaging of platelets according to claim 1, characterized in that the LED light source is a narrowband LED light source having a central wavelength within a visible light region and a bandwidth of 5 nm to 10 nm; or, the LED light source is a light source formed by coupling a broadband LED light source with a single-mode optical fiber, the broadband LED light source having a central wavelength within the visible light region and a bandwidth of 10 nm to 35 nm, and the single-mode optical fiber having a diameter of 30 μm to 250 μm.

8. A detection method based on the shadow imaging of platelets, comprising following detection steps:
step 1: fixing a microfluidic chip to the surface of an image sensor chip to form a sample chamber, and injecting a proper amount of human blood sample to be detected into the sample chamber;
step 2: illuminating the human blood sample placed in the sample chamber by using an LED light source as a lighting source of a shadow imaging device, and optically projecting and/or photographing the blood sample to be detected directly by the image sensor chip to obtain a projected image of the human blood sample, the physical pixel size occupied by a directly-projected image of abnormally-sized platelets on the image sensor chip being about 8 μm to 25 μm, and the physical pixel size occupied by a directly-projected image of normally-sized platelets being about 2 μm to 5 μm; and
step 3: statistically analyzing the shadow imaging results of the human blood sample in the step 2, and obtaining the number and proportion of the abnormally-sized platelets in the human blood sample in unit volume directly by an image processing algorithm according to the shadow imaging results since the physical pixel size occupied by the abnormally-sized platelets is significantly greater than the physical pixel size occupied by the normally-sized platelets.

9. The detection method based on the shadow imaging of platelets according to claim 8, characterized in that the human blood sample to be detected is platelet suspension and diluent thereof obtained by separation of human whole blood.

10. The detection method based on the shadow imaging of platelets according to claim 8, characterized in that the injection of the human blood sample to be detected into the sample chamber is manual injection using a pipette or an injector, or automatic injection using an injection pump.
